SEQUENCE LISTING (45 sequences)

SEQ ID NO: 1 - DNA, 263 bp, Homo sapiens
ttttcttttccctggtgtgtgaggaagcttgagatccaaaatgggactgccagggaaccagccttcgtggggcttggaccattttcgtcttctcattttcttttcgcgcccacgctgcgaggtaaatagcccctttcctggtccgggagccgaggggtgtgggaaagggaaaggacagtggtgggaggcgcagggaagagggcggtttggtttggaaaagtgcagcccgagagggagcagcaggctttggagcaaggtaaagt

SEQ ID NO: 2 - DNA, 40 bp, Homo sapiens
gaaaggacagtggtgggaggcgcagggaagagggcggttt

SEQ ID NO: 3 - DNA, 26 bp, Artificial
  primer RASSF1 forward
taattgccaatgaggaaaggggaagt

SEQ ID NO: 4 - DNA, 24 bp, Artificial
  primer RASSF1 reverse
ccgcaaccgttaagactgaaacgt

SEQ ID NO: 5 - DNA, 24 bp, Artificial
  primer MLH1 forward
ccatgcactggtatacaaagtccc

SEQ ID NO: 6 - DNA, 20 bp, Artificial
  primer MLH1 reverse
gatgcgctgtacatgcctct

SEQ ID NO: 7 - DNA, 27 bp, Artificial
  primer MSH2 forward
gccttgcagctgagtaaacacagaaag

SEQ ID NO: 8 - DNA, 27 bp, Artificial
  primer MSH2 reverse
gtgcctccgcactggagaggctgctca

SEQ ID NO: 9 - DNA, 26 bp, Artificial
  left primer PTPRG intron 1
gatttaaaatgggattgttagggaat

SEQ ID NO: 10 - DNA, 27 bp, Artificial
  right primer PTPRG intron 1
cttactccaaaacctactactccctct

SEQ ID NO: 11 - DNA, 21 bp, Artificial
  primer PTPRG_L
gaaaggacagtggtgggaggc

SEQ ID NO: 12 - DNA, 18 bp, Artificial
  primer PTPRG_R
gcagggaagagggcggtt

SEQ ID NO: 13 - DNA, 24 bp, Artificial
  primer BRCA2-L
ggccatggaatctgctgaacaaaa

SEQ ID NO: 14 - DNA, 45 bp, Artificial
  primer BRCA2_R
ggaacaaggtttatcaagggatgtcacaaccgtgtggaagttgcg

SEQ ID NO: 15 - DNA, 628 bp, Homo sapiens
atgggtgcagtgggaggtcgtggagggctgccttgcaaattagaggaatttccaattccacttctgtccttcttgcgctcggggatgcagtcagtgggctgaccgtggttttcttttccctggtgtgtgaggaagcttgagatccaaaatgggactgccagggaaccagccttcgtggggcttggaccattttcgtcttctcattttcttttcgcgcccacgctgcgaggtaaatagcccctttcctggtccgggagccgaggggtgtgggaaagggaaaggacagtggtgggaggcgcagggaagagggcggtttggtttggaaaagtgcagcccgagagggagcagcaggctttggagcaaggtaaagttaaaatatcagagctctgggagacgctggcttttcgttttccgaggttgccgcgacgccgctggacctcagggggcgcccccgagccactggtggggctcctgccacctccacactggtcggcctcggccacctccacgcctcagggatggggcgcgcgtgcccgggttcccaggctctggggctgcagacgcgccttggcgcgaggcggcggcctcggcctggcgatgctgctcctggctttctcgaaacccgctg

SEQ ID NO: 16 - DNA, 26 bp, Artificial
  primer jb_47B02bsaL
gatttaaaatgggattgttagggaat

SEQ ID NO: 17 - DNA, 27 bp, Artificial
  primer jb_47B02bsaR
cttactccaaaacctactactccctct

SEQ ID NO: 18 - DNA, 24 bp, Artificial
  primer PTPRG_BSAS1_FW
tctcaaacttcctcacacaccaaa

SEQ ID NO: 19 - DNA, 22 bp, Artificial
  primer PTPRG_BSAS1_RV2
gtggagggttgttttgtaaatt

SEQ ID NO: 20 - DNA, 22 bp, Artificial
  primer PTPRG2_BSAS2_FW
agagggagtagtaggttttgga

SEQ ID NO: 21 - DNA, 20 bp, Artificial
  primer PTPRG2_BSAS2_RV
aaaaaccccaccaataactc

SEQ ID NO: 22 - DNA, 36 bp, Artificial
  primerPTPRG_FW_MS
aggaagagaggatttaaaatgggattgttagggaat

SEQ ID NO: 23 - DNA, 58 bp, Artificial
  primer PTPRG_RV_MS
cagtaatacgactcactatagggagaaggctcttactccaaaacctactactccctct

SEQ ID NO: 24 - DNA, 23 bp, Artificial
  primer PTPRG_MSP1_M_Left
gggatgtagttagtgggttgatc

SEQ ID NO: 25 - DNA, 22 bp, Artificial
  primer PTPRG_MSP1_M_Right
tatttacctcgcaacgtaaacg

SEQ ID NO: 26 - DNA, 23 bp, Artificial
  primer PTPRG_MSP1_U_Left
gggatgtagttagtgggttgatt

SEQ ID NO: 27 - DNA, 29 bp, Artificial
  primer PTPRG_MSP1_U_Right
aaaaactatttacctcacaacataaacac

SEQ ID NO: 28 - DNA, 19 bp, Artificial
  primer PTPRG_MSP2_M_Left
tttttcgcgtttacgttgc

SEQ ID NO: 29 - DNA, 22 bp, Artificial
  primer PTPRG_MSP2_M_Right
ccctctcgaactacacttttcc

SEQ ID NO: 30 - DNA, 22 bp, Artificial
  primer PTPRG_MSP2_U_Left
tttttttttgtgtttatgttgt

SEQ ID NO: 31 - DNA, 23 bp, Artificial
  primer PTPRG_MSP2_U_Right
tccctctcaaactacacttttcc

SEQ ID NO: 32 - DNA, 25 bp, Artificial
  primer PTPRG_MSP3_M_Left
aaaatattagagttttgggagacgt

SEQ ID NO: 33 - DNA, 22 bp, Artificial
  primer PTPRG_MSP3_M_Right
acgtaaaaataaccgaaaccga

SEQ ID NO: 34 - DNA, 25 bp, Artificial
  primer PTPRG_MSP3_U_Left
aaaatattagagttttgggagatgt

SEQ ID NO: 35 - DNA, 25 bp, Artificial
  primer PTPRG_MSP3_U_Right
aaacataaaaataaccaaaaccaac

SEQ ID NO: 36 - DNA, 45 bp, Artificial
  primer PTPRG_MLPA1_Left
gggttccctaagggttggagaggtaaatagcccctttcctggtcc

SEQ ID NO: 37 - DNA, 41 bp, Artificial
  primer PTPRG_MLPA1_Right
gggagccgaggggtgtgttctagattggatcttgctggcgc

SEQ ID NO: 38 - DNA, 40 bp, Artificial
  primer PTPRG_MLPA2_Left
gggttccctaagggttggagaaaggacagtggtgggaggc

SEQ ID NO: 39 - DNA, 42 bp, Artificial
  primer PTPRG_MLPA2_Right
gcagggaagagggcggttttctagattggatcttgctggcgc

SEQ ID NO: 40 - DNA, 48 bp, Artificial
  primer PTPRG_MLPA3_Left
gggttccctaagggttggaccattttcgtcttctcattttcttttcgc

SEQ ID NO: 41 - DNA, 42 bp, Artificial
  primer PTPRG_MLPA3_Right
gcccacgctgcgaggtaaatctagattggatcttgctggcgc

SEQ ID NO: 42 - DNA, 50 bp, Artificial
  primer PTPRG_MLPA4_Left
gggttccctaagggttggataatttccaattccacttctgtccttcttgc

SEQ ID NO: 43 - DNA, 44 bp, Artificial
  primer PTPRG_MLPA4_Right
gctcggggatgcagtcagtgttctagattggatcttgctggcgc

SEQ ID NO: 44 - DNA, 19 bp, Artificial
  universal primer MLPA_F
gggttccctaagggttgga

SEQ ID NO: 45 - DNA, 23 bp, Artificial
  universal primer MLPA_R
gcgccagcaagatccaatctaga

(19) United States
(12) Patent Application Publication
(10) Pub. No.: US 2011/0053149 A1
(43) Pub. Date: Mar. 3, 2011

(54) METHYLATION DETECTION IN THE GENOMIC REGION OF A RECEPTOR PROTEINTYROSINE PHOSPHATASE GAMMA GENE FOR DETECTION AND/OR DIAGNOSIS OF A TUMOUR

(76) Inventors: Judith Mary Boer, Haarlem (NL); Eddy Herman Jaspar Van Roon, Aan Den Rijn (NL); Johannes Morreau, Bloemendaal (NL)

(21) Appl. No.: 12/735,260

(22) PCT Filed: Dec. 29, 2008
(86) PCT No.: PCT/NL2008/050857

§ 371 Date: Oct. 4, 2010

(30) Foreign Application Priority Data
Dec. 28, 2007 (EP) 07150458.3

(51) Int. Cl. C12Q 1/68

(52) U.S. Cl. 435/6

(57) ABSTRACT

The invention discloses means and methods for detecting methylation of a CpG in a region of a receptor protein tyrosine phosphatase gamma gene in samples obtained from a relevant site of an individual. The means and methods can be used to determine whether the individual is suffering from a tumor.

### TECHNICAL FIELD

The invention relates to the field of medicine and diagnosis. In particular, the invention relates to methylation of genomic DNA and the correlation with the presence of cancer cells or precursors thereof in a sample. The invention further relates to the use of the genomic region of receptor protein-tyrosine phosphatase gamma gene therein and the use of the genomic region to screen for CpG methylation involved in the early stages of tumorigenesis.

### BACKGROUND

The development of cancerous lesions is caused by the acquisition of a series of changes in the DNA that give a cell a selective growth advantage. These changes can be genetic, such as point mutations or deletions, or epigenetic, such as DNA methylation and histone modifications. The main epigenetic modification in mammals is methylation of cytosine residues of CpG dinucleotides, which can alter the expression of genes and is transmitted through cell division. The proximal promoter and first exon regions of 40-50% of human genes contain small (0.5 to several kb) clusters of CpGs, called CpG islands (CGIs) that are protected from methylation [Bird (1986), Nature 321:209-13]. This lack of methylation might be a prerequisite for active transcription, as illustrated by two normal exceptions to this situation. Fully methylated CGIs are found only in promoters of silenced alleles for selected imprinted autosomal genes [Li (1993), Nature 366:362-5] and multiple silenced genes on the inactivated X-chromosomes of females [Mohandas et al. (1981), Science 211:393-6; and Surani (1998), Cell 93:309-12]. Cancer susceptibility may be influenced by differences in stringency of epigenetic control.

Tumors are often characterized by an imbalance in cytosine methylation as manifested both by regional hypermethylation of CGIs and by global hypomethylation [Ehrlich (2002), Oncogene 21:5400-13; Gama-Sosa et al. (1983), Nucl. Acids Res. 11:6883-94; Goelz et al. (1985), Science 228:187-90; and Feinberg et al. (1988), Cancer Res. 48:1159-61]. The role of aberrant DNA methylation in cancer is well documented. A growing number of cancer genes are being recognized that harbor dense methylation in normally unmethylated promoter CGIs [Jones and Laird (1999), Nat. Genet. 21:163-7]. This methylation both marks and plays a key role in an epigenetically mediated loss-of-gene function [Baylin and Herman (2000), Trends Genet. 16:168-74]. Almost half of the tumor-suppressor genes that cause familial cancers through germline mutations can be inactivated in association with promoter hypermethylation in sporadic cancers, including Rb, APC, VHL, p16INK4A, BRCA1, E-cadherin and hMLH1 [Jones and Laird (1999), Nat. Genet. 21:163-7; and Baylin and Herman (2000), Trends Genet. 16:168-74]. Hypermethylation of hMLH1 causes microsatellite instability (MSI) in sporadic cancers, including colorectal cancer (CRC) [Herman et al. (1998), Proc. Natl. Acad. Sci. U.S.A. 95:6870-5; Veigl et al. (1998), Proc. Natl. Acad. Sci. U.S.A. 95:8698-702]. In addition to classic tumor-suppressor genes, promoter hypermethylation is being associated with a growing list of other genes that have strongly been implicated in tumorigenesis and for which loss of function appears to be linked primarily with this epigenetic mode of inactivation [Baylin and Herman (2000), Trends Genet. 16:168-74].

Some tumor types have a higher percentage of methylated known CGIs than others: for example, the most hypermethylated tumors originate from the gastrointestinal tract (oesophagus, stomach, colon), while significantly less hypermethylation has been reported in, e.g., ovarian tumors [Esteller et al. (2001), Cancer Res. 61:3225-9]. It has become clear that CGI hypermethylation is not restricted to a few CGIs, but affects multiple loci and exists in a gradual range across cancer cell lines [Paz et al. (2003), Cancer Res. 63:1114-21] and primary human tumors [Esteller et al. (2001), Cancer Res. 61:3225-9]. Therefore, it is more appropriate to perform more refined methylation profiling rather than stratification into CGI methylator positive (CIMP+) or negative (CIMP−) phenotypes based on only a few loci [Whitehall et al. (2002), Cancer Res. 62:6011-4; Suter et al. (2003), Br. J. Cancer 88:413-9; Toyota et al. (1999), Proc. Natl. Acad. Sci. U.S.A. 96:8681-6]. Several approaches are now available for profiling CGI methylation in human cancers. Microarray-based approaches have the advantage of being technically simple; they do not require large amounts of DNA, and they can screen thousands of loci in parallel [Ushijima (2005), Nature Reviews Cancer 5:223-231]. Huang and co-workers have developed an array-based strategy containing short GC-rich tags for differential methylation hybridization [Huang et al. (1999), Hum. Mol. Genet. 8:459-70]. Tumor DNA and normal DNA are each digested with methylation-sensitive restriction enzymes, and PCR amplicons derived from each sample are then hybridized to a CGI microarray. Using a panel of around 8000 CGIs, primary tumors could be hierarchically clustered into groups based on their methylation profiles that correlated with histological grade [Yan et al. (2000), Clin. Cancer Res. 6:1432-8] and hormone receptor status [Yan et al. (2001), Cancer Res. 61:8375-80] in breast tumors, with progression-free survival in late-stage ovarian cancer [Wei et al. (2002), Clin. Cancer Res. 8:2246-52], and with subtypes of cutaneous T-cell lymphomas [van Doom et al. (2005), J. Clin. Oncol. 23:3886-96].

Several studies have been performed describing the relationship between CGI methylation and CRC [e.g., Toyota et al. (1999), Proc. Natl. Acad. Sci. U.S.A. 96:8681; Bai et al. (2004), Int. J. Cancer 112:846-853; Rashid et al. (2001), Am. J. Pathol. 159:1129-35]. Using MSP to determine the methylation status of 30 new MINT loci and three known tumor-suppressor genes in primary CRCs and adenomas, Toyota and co-workers [Toyota et al. (1999), Proc. Natl. Acad. Sci. U.S.A. 96:8681] found that the majority of CGI methylation events in CRCs are age-related. Virtually all the other methylation events occurred in a distinct subset of CRCs and adenomas that they termed CGI methylator phenotype positive (CIMP+). Similarly, other studies demonstrated the early and specific involvement of promoter hypermethylation of several tumor-related genes in the colorectal adenoma-carcinoma sequence [Bai et al. (2004), Int. J. Cancer 112:846-853; Rashid et al. (2001), Am. J. Pathol. 159:1129-35].

The distinction between CIMP+ and CIMP− arises very early in the pathogenesis of CRC [Kondo and Issa (2004), Cancer and Metastasis Reviews 23:29-39], possibly as early as the aberrant crypt foci. Aberrant methylation also contributes to later stages of colon cancer formation and progression [Kondo and Issa (2004), Cancer and Metastasis Reviews 23:29-39] and has been related to the serrated pathway [Jass et al. (2000), Histopathology 37:295-301]. The CIMP+tumors, comprising half of all sporadic CRC, are distinctly characterized by pathology, clinical and molecular genetic features. Genetically, CIMP+CRCs can be divided into two groups; one including the majority of sporadic MSI-High cancers related to hMLH1 promoter methylation [Toyota et al. (1999), Proc. Natl. Acad. Sci. U.S.A. 96:8681], and another with a high incidence of K-ras mutations and a low incidence of p53 mutations [Whitehall et al. (2001), Cancer Res. 61:827-30; Toyota et al. (2000), Proc. Natl. Acad. Sci. U.S.A. 97:710-5]. Part of the association of CIMP+ with K-ras mutations may be related to silencing of the DNA repair gene MGMT by promoter methylation, which has been reported to increase the incidence of G-A mutations [Esteller et al. (2000), Cancer Res. 60:2368-71]. CIMP+can coexist with APC mutations in sporadic CRC [Hawkins et al. (2002), Gastroenterology 122:1376-87; Gayet et al. (2001), Oncogene 20:5025-32], and is also found in MSI-stable CRC and cell lines [Whitehall et al. (2002), Cancer Res. 62:6011-4; Suter et al. (2003), Br. J. Cancer 88:413-9]. These findings suggest that hypermethylation may be involved in both colorectal tumorigenesis pathways.

Suzuki et al. performed a genomic screen for genes upregulated by demethylation and histone deacetylase inhibition in a human CRC cell line. Subsequent analysis of paired primary CRC tumors and normal tissues showed an association with hypermethylated 5′ CGIs in a tumor-specific manner [Suzuki et al. (2002), Nat. Genet. 31:141-9]. These results are promising for future studies into the biological mechanisms and clinical consequences of CGI methylation in CRC. What are still lacking are highly informative methylation markers for cancer detection including its precursor forms and the association of methylation patterns with clinical behavior of CRC. This requires the methylation profiling of large numbers of well-defined tumors with known clinical outcome.

The invention now provides a method for determining whether an individual is suffering from a tumor in a tissue, comprising determining from a sample comprising nucleic acid from the tissue, the methylation of a CpG in the genomic region of a receptor protein-tyrosine phosphatase gamma gene and determining from the methylation whether the individual is suffering from a tumor in the tissue. Methylation of a CpG in the genomic region of a receptor protein-tyrosine phosphatase gamma gene (protein tyrosine phosphatase, receptor type, G; PTPRG, Ref seq. ID NC_{—}000003) can be used as an early marker for determining whether an individual is suffering from a tumor in a tissue.

The term “tumor” refers to an abnormal increase in the number of cells that has developed in a tissue. A tumor can be benign and not metastasized. Non-limiting examples of a benign tumor are prostatic hyperplasia, cutaneous lymphoid hyperplasia, colorectal polyps or adenomas, and benign ductal or lobular hyperplasia of the breast. While some tumors are not associated with an increased risk of progression into a malignant and/or metastasized tumor, often, however, a tumor is associated with an increased risk of malignant transformation. Non-limiting examples of this type of tumors are C-cell hyperplasia, a premalignant stage in the development of medullary thyroid carcinoma, colorectal polyps or adenomas, and atypical ductal or lobular tumor of the breast, which presence indicates an increased risk of developing cancer.

The term “tumor” also refers to malignant primary or metastasized tumors typically referred to as neoplasia. Examples thereof include, but are not limited to, a carcinoma, a sarcoma, a lymphoma, a leukemia, or a myeloma.

A tumor can be present in any tissue or part of a body including, but not limited to, bone, brain, eye, breast, skin, bladder, lung, ureter, thyroid, parathyroid, salivary gland, kidney, prostate, genital system including ovary and testis, endometrium, blood/hematologic system, or in a gastrointestinal tissue. In a preferred embodiment, the tumor is an epithelial tumor or at least of epithelial origin.

In a preferred embodiment, a “sample comprising nucleic acid from the tissue” refers to a sample comprising nucleic acid, preferably desoxyribonucleic acid, from epithelial tumor cells.

In a preferred embodiment, the tumor is present in a gastrointestinal tissue. The gastrointestinal system comprises organs such as mouth, oesophagus, stomach, small and large intestine, anus, liver, bile duct, and pancreas. Tumors of a gastrointestinal tissue comprise a polyp or adenoma, gastrointestinal stromal tumor, gastrointestinal sarcoma, gastrointestinal mesenchymal tumor, leiomyoma, leiomyosarcoma, leiomyoblastoma, and gastrointestinal carcinoma.

A tumor of the gastrointestinal system can be a polyp or adenoma. Polyps are typically of epithelial origin and can be found in tissues comprising a mucous membrane such as colon, small intestine, stomach, nose, urinary bladder, cervix and uterus. The polyp can be an inflammatory polyp, a hyperplastic polyp or an adenomatous polyp. Although most polyps themselves are benign, the presence of a polyp is indicative of an increased risk for developing cancer. Adenomatous polyps can be divided into villous, tubular and tubulovillous adenomatous polyps.

A tumor of the gastrointestinal system can also be a carcinoma, a cancer of epithelial tissue that covers or lines surfaces of organs, glands, or body structures. The carcinoma can be an adenocarcinoma, or an undifferentiated carcinoma.

The transformation of normal gastrointestinal epithelial cells to cancer cells follows a process of molecular and histological changes. The drivers of this process are genetic and epigenetic alterations, leading to growth advantages and expansion of the altered cells. The transformation from a normal epithelial cell to a polyp to a carcinoma occurs over a period of about ten years, whereby histological changes occur at each step in this process starting with a benign tubular adenoma to an invasive adenocarcinoma.

In a further preferred embodiment, the tumor is present in colorectal tissue. Colorectal tumors are the fourth most often diagnosed tumor in both male and female, accounting to about 10% of all cancer deaths. Cancer of the colon is highly treatable and often curable by surgery. However, local recurrence or recurrence at a distant site following surgery occurs in about 50% of all cases. Patients of which the tumor had penetrated beyond the bowel wall and/or there was evidence of metastasis to distant organs at the moment of surgery, have a five year survival rate of less than ten percent. In general, early diagnosis and treatment of a colorectal tumor enhances the survival rate, as this limits the chance of recurrence of the tumor.

A few hereditary diseases are known that increase the risk for developing a tumor of the gastrointestinal system. These diseases include familial adenomatous polyposis, a rare genetic disease in which people develop tumors of the adenomatous type in the colon and often also in the upper intestine; Gardner's syndrome, causing tumors to develop throughout the colon and upper intestine and also in other parts of the body such as skin (sebaceous cysts and lipomas), bone (osteomas) and abdomen (desmoids); MUTYH-associated polyposis, a rare autosomal recessive disease caused by mutations in the MUTYH gene, the human homologue of the Escherichia coli mutY gene; and hereditary nonpolyposis colorectal cancer, causing not only tumors in the colon but also in other organs. Hereditary nonpolyposis colorectal cancer includes Lynch I and Lynch II syndromes. Lynch I syndrome usually leads to the development of a small number of polyps that quickly become malignant. Lynch II syndrome often leads to the development of tumors in the breast, stomach, small intestine, urinary tract and ovaries as well as in the colon. Thus, in a preferred embodiment, the individual is suspected or diagnosed as hereditary at risk of developing a tumor of the gastro-intestinal tract.

Methylation of a CpG in the genomic region of a gene often results in transcriptional silencing of the gene through complex effects on transcription factor binding and associated changes in chromatin structure. These effects typically, though not necessarily, involve methylation of CpGs in promoter/enhancer and/or other transcriptional regulatory sequences. Aberrant methylation may play a role in the transformation process of cells by silencing genes that normally prevent growth of cells. Methylation of CpG can also affect other phenomena in a cell. Sequences that are involved in these phenomena typically, though not necessarily, reside within a 1 mega base pairs of the genomic sequence of the gene they affect. Following this general theme, it is thought that methylation of a CpG in a region more than 1 mega base pairs upstream from a transcription initiation site or more than 1 mega base pairs downstream from a poylyadenylation site are less likely to be genetically linked to allow adequate assessment and/or diagnosis of the risk that the individual has for having and/or developing a tumor of the tissue.

In a preferred embodiment, therefore, the genomic region of the receptor protein-tyrosine phosphatase gamma gene is defined herein as a region from 1 mega base pairs upstream from the most upstream transcription initiation site of the gene to 1 mega base pairs downstream from the most distant poylyadenylation site of the gene, more preferred from 100 kilo base pairs upstream from the most upstream transcription initiation site of the gene to 100 kilo base pairs downstream from the most distant poylyadenylation site of the gene, or most preferred from 10 kilo base pairs upstream from the most upstream transcription initiation site of the gene to 10 kilo base pairs downstream from the most distant poylyadenylation site of the gene.

In a preferred embodiment, one end of the region is a region from 1 mega base pairs upstream from the transcription initiation site at chr3:61,522,283 (March 2006 human reference sequence (NCBI Build 36.1)) to 1 mega base pairs downstream from the poylyadenylation site at chr3:62,255,613 of the gene, more preferred from 100 kilo base pairs upstream from the most upstream transcription initiation site of the gene to 100 kilo base pairs downstream from the most distant poylyadenylation site of the gene, or most preferred from 10 kilo base pairs upstream from the most upstream transcription initiation site of the gene to 10 kilo base pairs downstream from the most distant poylyadenylation site of the gene.

In a preferred embodiment, methylation of a CpG that is present in a first intron of the receptor protein-tyrosine phosphatase gamma gene is determined. Methylation of at least one CpG in the first intron was found to be an early marker for the presence of a colorectal tumor in an individual. The genomic region on chromosome 3p14.2 comprises two CpG-rich regions that are present within the intron (see FIG. 4). In a preferred method of the invention, methylation of at least one CpG from the genomic region as indicated in FIG. 4, preferably at least one CpG selected from CpG1, CpG2, CpG3, CpG4, CpG5, CpG6, CpG7, CpG8, CpG9, CpG 10, CpG11, CpG12, CpG13, CpG14, CpG15, CpG16, CpG17, CpG18, CpG19, CpG 20, CpG21, CpG22, CpG23, CpG24, CpG25, CpG26, CpG27, CpG28, CpG29, CpG 30, CpG31, CpG32, CpG33, CpG34, CpG35, CpG36, and CpG37 as indicated in FIG. 4, is determined.

The genomic region encompasses the region covered by CpG island clone 47B02. Thus, in a preferred embodiment, methylation of at least one CpG selected from CpG1, CpG2, CpG3, CpG4, CpG5, CpG6, CpG7, CpG8, CpG9, CpG 10, and CpG11 as indicated in FIG. 4 in the CpG island is determined. In a further preferred embodiment, CpG is selected from CpG7, CpG8, CpG9, and CpG10, as indicated in FIG. 4. In a further preferred embodiment, CpG comprises CpG9 and CpG10, as indicated in FIG. 4. Preferably, CpG comprises CpG9.

A further preferred method according to the invention comprises determining methylation of at least two of the CpGs indicated in FIG. 4, more preferred at least three, more preferred at least four, more preferred at least five, more preferred at least six, more preferred at least seven, more preferred at least eight, more preferred at least nine, more preferred at least ten, more preferred at least fifteen, more preferred at least twenty, more preferred at least thirty, and most preferred at least thirty-seven of the CpGs indicated in FIG. 4.

A sample according to the invention is preferably isolated from blood, stool, or urine from the individual. For routine testing, it is important that the sample containing nucleic acid can be withdrawn from the individual in a cost-effective and patient-compliant manner. Body secretes, such as blood, stool and urine, can easily be used for these testing. Other preferred samples that can be used include, but are not limited to, samples comprising skin, hair, saliva, cheek swab, or lung fluid. In a preferred embodiment, the sample is a biopsy of an epithelial tissue. In a particularly preferred embodiment, the sample is a stool sample. In a preferred embodiment, the sample comprises nucleic acid of gastrointestinal cells. It is preferred that the nucleic acid is derived from cells from the tissue. In a further preferred embodiment, the sample comprises nucleic acid of colon cells.

In a preferred embodiment, the methylation of CpG is determined by comparing to a reference. The reference can be a sample from an individual of which the presence or absence of a tumor has been previously determined. In a preferred embodiment, the reference is taken from a sample from an individual of which relevant data, comprising position and number of methylated CpG nucleotides have been stored in a database. The database can be present in an electronic storage device, such as, but not limited to, a computer or a server. It is further preferred that the database comprising the reference can be addressed to compare the position and number of methylated CpG nucleotides with the reference. In a preferred embodiment, the reference comprises at least an unmethylated DNA and/or a fully methylated DNA.

As the markers of the present invention detect early stages of tumorigenesis, it is possible to determine whether an individual has such early stages. A method of the invention is, therefore, very sensitive in that also early stages are detected. When combined with a marker for late stages of tumorigenesis, it may be possible to determine whether the individual comprises an early or a late stage tumor. Thus, in a preferred embodiment, a method of the invention further comprises determining in a sample of the individual the presence or absence of a marker for late stage tumorigenesis. The tissue can be determined and/or diagnosed to be free of tumor, to comprise early stage tumor, or to comprise late stage tumor.

Methylation of CpGs in a sample can be determined using a variety of methods. As also described in the examples herein, the methods include, but are not limited to, differential methylation hybridization, methylation-specific multiplex ligation-dependent probe amplification (MS-MLPA), and methods based on bisulphite modification of DNA including bisulphite sequencing, methylation-specific PCR (MSP) and quantitative variations thereof, methylation-sensitive single nucleotide primer extension (MS-SnuPE), combined bisulphite restriction analysis (COBRA), methylation-sensitive high resolution melting (MS-HRM), array-based methods such as CpG island-specific micro-arrays, and/or mass spectrometry analysis. Genes and/or loci that are affected by aberrant methylation appear to have significant potential to be used as molecular markers for colon tumors, as well as for a variety of other tumors.

A preferred method for determining methylation of a CpG according to the invention comprises use of methylation-sensitive restriction of the test nucleic acid. Some of the restriction enzymes that are currently available to the artisan are sensitive to methylation and either require methylation for cleavage of the target nucleic acid or vice versa only cleave the target nucleic acid when it is not methylated. Use of such sensitive restriction enzymes provide test nucleic acid that is cut at the designated target site or not, depending on the methylation state of the target site nucleic acid. The digested nucleic acid can be used directly as a probe or be probed, or it can first be amplified and subsequently used as a probe. In the latter case, when using amplification primers that flank the target site, one will obtain an amplificate if the target site is not cleaved by the enzyme, and vice versa no amplificate when the target site is cleaved. The resulting product, whether or not successfully amplified, can subsequently be used as a probe or be probed. Methylation-dependent restriction of the nucleic acid can be performed by using methylation-sensitive restriction enzymes including, but not limited to, BstUI, HpaII and HhaI. A preferred method for detection based on methylation-sensitive restriction enzymes comprises multiplex ligation-dependent probe amplification [Nygren et al. (2005), Nucl. Acids Res. 33:e128].

Therefore, in a preferred method according to the invention, methylation of CpG is determined by amplifying the nucleic acid before and after methylation-dependent restriction of the nucleic acid.

Another convenient method is provided by treating the nucleic acid with sodium bisulphite, which converts unmethylated cytosines to uracils, but leaves methylated cytosines unchanged. Methods based on bisulphite-converted DNA include bisulphite sequence analysis [Grunau et al. (2001), Nucl. Acids Res. 29:e65], detection of methylation using bead arrays [Bibikova et al. (2006), Genome Res. 16:383-93], MSP [Herman et al. (1996), Proc. Natl. Acad. Sci. U.S.A. 93:9821-6], methylation detection by mass spectrometry [Ehrich et al. (2005), Proc. Natl. Acad. Sci. U.S.A. 102:15785-90], Ms-SNuPE [Gonzalgo and Jones (1997), Nucl. Acids Res. 25:2529-2531], and MS-HRM [Wojdacz and Dobrovic (2007), Nucl. Acids Res. 35:e41].

Therefore, in another preferred method according to the invention, methylation of CpG is determined with a method comprising bisulphite modification of the nucleic acid.

In an alternative embodiment, the method comprises labeling of the amplified nucleic acid and hybridization of the labeled nucleic acid to a microarray comprising probes that are able to hybridize to the labeled nucleic acid. The presence and quantity of hybridization signals of the labeled nucleic acid to a probe on the microarray can be determined as is known to a skilled person and is dependent on the label that is used for the nucleic acid. The difference in hybridization signal before and after methylation-dependent restriction of the nucleic acid can be used to determine the methylation of a CpG in the nucleic acid. Alternatively, a difference in hybridization signal is determined between samples from an individual suffering from a tumor in a tissue, or suspected of suffering therefrom, and healthy individuals that are treated with methylation-sensitive restriction enzymes, and/or between samples from an individual suffering from a tumor in a tissue, or suspected of suffering therefrom, and an individual with a tumor.

Especially preferred are methods that allow processing of multiple samples in an economical and time-efficient way, such as MS-MLPA, custom bead arrays, and multiplex PCR methods based on pre-treatment with methylation-sensitive restriction enzymes [Nygren et al. (2005), Nucl. Acids Res. 33:e128] or based on pre-treatment with bisulphite such as MSP and quantitative derivatives thereof such as quantitative multiplex-MSP [QM-MSP; Fackler et al. (2004), Cancer Research 64:4442-4452].

In a particularly preferred embodiment, a methylation-specific multiplex ligation-dependent probe amplification (MS-MLPA) assay is used to test methylation of specific CpGs in the 3′ region of the BSA-validated region in a consecutive CRC validation series (FIG. 11). The MS-MLPA assay is robust and can be performed on DNA derived from formalin-fixed paraffin-embedded tissues.

The invention furthermore provides a kit for determining whether a person suffers from a tumor, the kit comprising means for determining the methylation of a CpG present in a genomic region of a receptor protein-tyrosine phosphatase gamma gene in a sample comprising nucleic acid from the person, the genomic region including a region up to 1 mega base pairs upstream from the most upstream transcription initiation site and 1 mega base pairs downstream from the most distal polyadenylation site of the gene, more preferred from 100 kilo base pairs upstream from the most upstream transcription initiation site of the gene to 100 kilo base pairs downstream from the most distant poylyadenylation site of the gene, or most preferred from 10 kilo base pairs upstream from the most upstream transcription initiation site of the gene to 10 kilo base pairs downstream from the most distant poylyadenylation site of the gene.

Methods to isolate nucleic acid, such as desoxyribonucleic acid, from stool are known in the art and comprise “QIAamp DNA Stool Mini Kit” (Qiagen, the Netherlands) and “PSP® Spin Stool Genomic DNA Purification Kit” (Invitek, Germany).

A kit according to the invention preferably comprises at least two primers that allow amplification of the genomic region comprising CpG. Amplification can be performed by any method known in the art including, but not limited to, polymerase chain reaction, strand displacement amplification, nucleic acid sequence-based amplification, rolling circle amplification technology, and transcription-mediated amplification. Each of these amplification methods uses different approaches to achieve the amplification of nucleic acid molecules to amounts that can subsequently be detected. The kit preferably also comprises means for treating the DNA with bisulphite, or means for treating the DNA with a relevant methylation-sensitive restriction enzyme, prior to amplification.

In a preferred embodiment, a kit according to the invention provides means for amplifying a CpG that is present in a first intron of the receptor protein-tyrosine phosphatase gamma gene. In this embodiment, a set of primers can be used that allow amplification of the first intron, or at least a part of the intron that comprises a CpG marker. A preferred set of primers is selected from the primers provided in FIG. 8. Preferably, the set of primers is a set provided in FIG. 8C. Particularly preferred is the set indicated by MLPA2 in FIG. 8C.

The invention also provides the use of a kit according to the invention for determining whether an individual is suffering from a tumor in a tissue. The use of a kit according to the invention provides a cost-effective and patient-compliant way of using an early marker for prognosing or diagnosing an individual for the presence of a tumor.

In another embodiment, the invention provides a method for determining whether an individual is suffering from a tumor in a tissue, comprising determining from a sample containing nucleic acid from the tissue an mRNA expression level of the receptor protein-tyrosine phosphatase gamma gene and determining from the expression level whether the individual is suffering from a tumor in the tissue.

The mRNA expression level of the receptor protein-tyrosine phosphatase gamma gene can be determined by any method known to a skilled person including, but not limited to, Northern blotting and quantitative reverse transcriptase-PCR.

In yet another embodiment, the invention provides a method for determining whether an individual is suffering from a tumor in a tissue, comprising determining from a sample containing protein from the tissue a protein expression level of the receptor protein-tyrosine phosphatase gamma gene and determining from the expression level whether the individual is suffering from a tumor in the tissue.

The protein expression level of the receptor protein-tyrosine phosphatase gamma gene can be determined by any method known to a skilled person including, but not limited to, Western blotting and immunohistochemistry.

In a preferred embodiment, the method comprises comparing the determined expression level of the receptor protein-tyrosine phosphatase gamma gene to the expression level of the gene in a reference sample.

The invention further provides a method for determining whether an individual is suffering from a tumor in a tissue, comprising determining in a sample comprising nucleic acid from the tissue, the methylation of a binding site for CCCTC-binding factor (zinc finger protein), also called CTCF, in the genomic region of the receptor protein-tyrosine phosphatase gamma gene and determining from the methylation whether the individual is suffering from a tumor in the tissue.

The invention further provides a method for determining whether an individual is suffering from a tumor in a tissue, comprising determining in a sample comprising nucleic acid of the first intron of the receptor protein-tyrosine phosphatase gamma gene from the tissue, whether the CTCF protein, can bind to the nucleic acid of the first intron. Also provided is the use of CTCF protein for determining whether a sample of a tissue of an individual comprises tumor cells. Further provided is a method for determining whether a sample of a tissue of an individual comprises tumor cells comprising determining whether CTCF protein can bind to nucleic acid of the first intron of the receptor protein-tyrosine phosphatase gamma gene from the tissue.

The term “CTCF” refers to a protein involved in insulator activity or to a nucleotide coding for the protein. The gene has the Ref seq. ID NC_{—}000016. The protein CTCF plays among others a role of repressing the insulin-like growth factor 2 gene, by binding to the H-19 imprinting control region (ICR) along with Differentially-methylated Region-1 (DMR1) and MAR3.

Binding of targeting sequence elements by CTCF can block the interaction between enhancers and promoters, therefore, limiting the activity of enhancers to certain functional domains. Besides acting as an enhancer blocker, CTCF can also act as a chromatin barrier by preventing the spread of heterochromatin structures.

Two independent studies (Xie et al. from MIT and Kim et al. from UCSD) revealed that the human genome contains nearly 15,000 CTCF insulator sites, suggesting a wide-spread role of CTCF in gene regulation (X. Xie, T. S. Mikkelsen, A. Gnirke, K. Lindblad-Toh, M. Kellis, and E. S. Lander (2007), “Systematic discovery of regulatory motifs in conserved regions of the human genome, including thousands of CTCF insulator sites,” Proc. Natl. Acad. Sci. U.S.A. 104(17):7145-50. doi:10.1073/pnas.0701811104. PMID 17442748.

T. H. Kim, Z. K. Abdullaev, A. D. Smith, K. A. Ching, D. I. Loukinov, R. D. Green, M. Q. Zhang, V. V. Lobanenkov, and B. Ren (2007), “Analysis of the vertebrate insulator protein CTCF-binding sites in the human genome,” Cell 128(6):1231-45).

It was further revealed that CTCF binding sites act as nucleosome positioning anchors so that, when used to align various genomic signals, multiple flanking nucleosomes can be readily identified (Y. Fu, M. Sinha, C. L. Peterson, and Z. Weng (2008), “The insulator binding protein CTCF positions 20 nucleosomes around its binding sites across the human genome,” PLoS Genetics 4(7):e1000138. doi:10.1371/journal.pgen.1000138. PMID 18654629).

In a preferred embodiment, the CTCF binding site is a CTCF binding site in the region OREG0015647, chr3: 61,525,101-61,525851 of UCSC March 2006 assembly. In a more preferred embodiment, the CTCF binding site comprises the DNA sequence:

(SEQ ID NO: 1)
ttttcttttccctggtgtgtgaggaagcttgagatccaaaatgggactgc
cagggaaccagccttCG¹tggggcttggaccattttaCG²tcttctcatt
ttcttttCG³CG⁴cccaCG⁵ctgCG⁶aggtaaatagcccctttcctggtc
CG⁷ggagcCG⁸aggggtgtgggaaagggaaaggacagtggtgggaggCG⁹
cagggaagagggCG¹⁰gtttggtttggaaaagtgcagccCG¹¹agaggga
gcagcaggctttggagcaaggtaaagt.

In an even more preferred embodiment, the CTCF binding site comprises the DNA sequence: gaaaggacagtggtgggaggCG⁹cagggaagagggCG¹⁰gttt (SEQ ID NO:2).

Methods of determining whether a protein can bind to DNA are known in the art. A preferred method is by performing chromatin immunoprecipitation with an antibody against the CTCF binding site. Preferably, methylation of the CTCF binding site is determined, preferably using a restriction enzyme specific for the methylation-sensitive HhaI site. In a more preferred embodiment, a CTCF binding site obtained by immunoprecipitation is amplified. In a more preferred embodiment, the CTFC binding site is amplified using a primer according to FIG. 8C.

The invention further provides a method for determining whether methylation of a binding site for the CTCF protein in the genomic region of the receptor protein-tyrosine phosphatase gamma gene is correlated with the occurrence of a tumor in a tissue sample, the method comprising determining whether methylation of a CpG in the genomic region is correlated with the occurrence of the tumor and determining whether the methylation state of the CpG affects the binding of CTCF to the nucleic acid of the genomic region of the receptor protein-tyrosine phosphatase gamma gene. In a preferred embodiment, the CTCF binding is determining in a nucleic acid of about 50 nucleotides of the genomic region comprising the CpG.

### BRIEF DESCRIPTION OF THE DRAWINGS

FIG. 1. Methylation profiling by differential methylation hybridization on CpG island clone microarrays detects differentially methylated loci. P-value curve for ANOVA results after testing for differences in methylation ratios between three histology groups: carcinoma, adenoma, normal. The red dotted line indicates the multiple testing corrected p-value cutoff of 0.0001. At this cutoff, 20 loci were selected, the most significant one clone 47B02.

FIG. 2. The 47B02 locus is hypermethylated in tumors. Variance plot of the ANOVA in FIG. 1, showing the log 10 ratios of the grouped samples for the PTPRG intron 1 locus. The log ratios of tumor and adenoma samples compared to the tumor cell line reference panel are close to 0, compared to a negative log ratio for the normals.

FIG. 3. Methylation profiles of 20 selected loci. Trend plot of the “top-20” differentially methylated loci (see FIG. 1), showing the log 10 ratios in the normal, adenoma and carcinoma hybridizations. As all of the loci have a lower log 10 ratio in the normal group (n=5) in comparison to the carcinoma group (n=17), one may conclude that these loci are hypermethylated in carcinomas. The log 10 ratios of the adenoma group (n=2) are all in the same order as the normal group, except for the PTPRG intron 1 clone (dark blue line).

FIG. 4. Genomic sequence encompassing CpG island clone 47B02 located at chr3:61,524,993-61,525,363 (UCSC assembly of March 2006) (shaded), and downstream CpG-rich region (chr3:61,525,384-61,525,620). All CpGs are indicated in red. The locations of one pair of primers for bisulfite sequence analysis are underlined. Note that the primer sequences are different, since they are based on DNA sequences after bisulfite modification, see FIG. 8A (BSA). The location of one MS-MLPA assay (MLPA2, see FIG. 8C) is double underlined. The 11 CpGs in the 47B02 sequence are in capitals and numbered 1-11. Restriction sites used for differential methylation hybridization amplicon generation are boxed: ccgg, HpaII; cgcg BstUI. The last 264 bp of the 47B02 sequence overlap with the first part of OREG0015647 (chr3:61,525,101-61,525,851). The start of the overlap is indicated by the * located four basepairs downstream of cg^{min 1} and continues 231 bp beyond the end of the sequence given here. (B) UCSC Genome Browser view of part of chromosome 3p (chr3: 61,524,969-61,525,930) showing part of intron 1 of PTPRG. Indicated are the relative size and location of CpG island clone 47B02 (blue), a small CpG island (light green), and regulatory element OREG0015647 from the OregAnno database (dark green; Kim et al. (2007), Cell 128:1231-45).

FIG. 5. Colon tumor-specific methylation of PTPRG intron 1 locus. The dot diagram indicates direct bisulfite sequencing results of ten CpG dinucleotides in 18 colon tumors (red bar) and 19 paired normal colon samples (green bar), using the BSA primers indicated in FIG. 8A. Black dot: methylated CpG; White dot: unmethylated CpG; Grey dot: sequence not readable. The black arrowheads indicate adenoma samples. On top are indicated the locations of the two methylation-sensitive restriction enzymes used in the amplicon generation for differential methylation hybridization to CpG island microarrays.

FIG. 6. Clonal bisulfite sequence analysis of PTPRG intron 1 locus confirmed direct bisulfite sequence results. The dot diagram indicates clonal bisulfite sequencing results of 10 CpG dinucleotides in (A) adenoma tID180 and (B) carcinoma tID127. N indicates a cloned allele from the normal tissue DNA, T for the tumor DNA. Black dot: methylated CpG; White dot: unmethylated CpG; Grey dot: sequence not readable. On top are indicated the locations of the two methylation-sensitive restriction enzymes used in the amplicon generation for differential methylation hybridization to CpG island microarrays.

FIG. 7. Bisulfite mass spectrometry analysis of PTPRG intron 1 locus. Representative example shown for carcinoma tID184 (80% tumor cells) and paired normal tissue. For the MS primers, the specific primer sequences were identical to the BSA primers and a tail was added to allow mass spectrometry application (see FIG. 8A). RNAse cleaved fragments are scored based on the shift in mass between a methylated fragment and an unmethylated fragment. The y-axis shows the percentage of methylated fragments in comparison with unmethylated ones. The values are the means and standard errors of three independent measurements. Fragments 1-3 contain CpGs 1-3, respectively, fragment 4 contains CpGs 4 and 5, fragment 5 contains CpGs 6 and 7, and fragments 6-9 contain one CpG each, i.e., CpGs 8-11.

FIG. 8. Primers for methylation detection of the PTPRG intron 1 locus. A. Bisulfite-sequence analysis (BSA) and methylation-specific mass spectrometry (MS). B. Methylation-specific PCR (MSP) on bisulfite modified DNA. C. Methylation-specific multiplex ligation-dependent probe amplification (MS-MLPA).

FIG. 9. Specificity and sensitivity of nine individual CpGs in the 47B02 region measured by direct bisulfite sequence analysis (see FIG. 5). Sensitivity indicates the percentage of tumors with methylation of the 47B02 genomic region. Specificity indicates the percentage of unmethylated normals.

FIG. 10. Distribution 0% and 100% methylated DNA controls. Cutoffs were based on controls included in ten individual experiments, including the fully methylated DNA (Chemicon-Millipore) and the unmethylated human semen DNA. Tested samples falling within three standard deviations of the mean of unmethylated and methylated control ratios were typed accordingly. Samples with ratios in between these boundaries were typed partially methylated.

FIG. 11. PTPRG intron 1 CpG9 methylation detected by MS-MLPA. Methylation frequency of PTPRG intron 1 CpG9 in carcinomas (Ca(T)), advanced adenomas (AA(T)) and corresponding normal epithelial tissue (CA(N) and AA(N)) as well as in precursor lesions hyperplastic polyps (HP), serrated adenoma (SA), early adenoma (EA). The number of tumors typed as methylated (dark), partially methylated (striped) and unmethylated (white) in the MS-MLPA assay is indicated.

FIG. 12. Sensitivity and specificity for PTPRG intron 1 CpG9 methylation in carcinomas and advanced adenomas.

### DETAILED DESCRIPTION OF THE INVENTION

### Examples

### Example 1

### DNA Samples

Anonymized tumor and normal colon mucosa biopsies were obtained and fresh-frozen. For isolation of DNA, pathologist-checked macrodissected (trimmed) frozen sections were used to minimize the percentage of normal epithelium and stromal cells. To control for patient-dependent methylation, we used normal epithelium from the same individuals as controls, where available. About 20 sections of 30 μm yielded at least 30-50 μg of DNA, which was sufficient for microarray hybridization and confirmation with alternative methods. Control DNA samples representing fully methylated (CpGenome universal methylated) and unmethylated (CpGenome universal unmethylated) DNA were obtained from Chemicon/Millipore. Colorectal carcinoma cell lines SW48, RKO, SW480, Caco2, SW837, and LS411 were obtained from the American Type Culture Collection (Manassas, US) and cultured according to the manufacturer's instructions. DNA was isolated using standard protocols [Isola et al. (1994), Am. J. Pathol. 145:1301-1308].

### CpG Island Microarrays

We obtained a copy of the 8600 CGI clone library from Dr. T. H. Huang (Center for Integrative Cancer Biology, The Ohio State University, Columbus, Ohio), based on a library originally generated by the Sanger Centre from affinity-purified in vitro methylated MseI-digested DNA fragments [Cross et al. (1994), Nat. Genet. 6:236-44]. The library was sequenced at the Toronto Microarray Facility. CGI clone inserts were amplified using vector-based primers essentially as described [Knijnenburg et al. (2005), Am. J. Med. Gen. 132A:36-40; Yan et al. (2002), Methods 27:162-9]. Three promoter regions of tumor-related genes were added, RASSF1 (forward TAATTGCCAATGAGGAAAGGGGAAGT (SEQ ID NO:3), reverse CCGCAACCGTTAAGACTGAAACGT (SEQ ID NO:4)), MLH1 (forward CCATGCACTGGTATACAAAGTCCC (SEQ ID NO:5), reverse GATGCGCTGTACATGCCTCT (SEQ ID NO:6)), and MSH2 (forward GCCTTGCAGCTGAGTAAACACAGAAAG (SEQ ID NO:7), reverse GTGCCTCCGCACTGGAGAGGCTGCTCA (SEQ ID NO:8)). The PCR products were filtered and spotted in 150 mM sodium phosphate buffer pH 8.5, 0.0002% sarkosyl onto CodeLink (GE Healthcare) slides using an OmniGrid arrayer (Genomic Solutions) as described [Knijnenburg et al. (2005), Am. J. Med. Gen. 132A:36-40].

### Differential Methylation Hybridization

The detection of differential methylation was based on methylation-sensitive restriction of MseI-digested, linker-ligated genomic DNA, followed by linker-mediated PCR amplification [Huang et al. (1999), Hum. Mol. Genet. 8:459-70]. Genomic fragments containing aberrantly methylated sites are protected from the digestion and can be amplified by linker-PCR, whereas the same fragments containing the unmethylated sites are cut and cannot be amplified. Sequential digestion with two methylation-sensitive endonucleases, HpaII and BstUI, enhances detection of CpG loci with extensive methylation in tumors and reduces the risk of incomplete digestion [Yan et al. (2001), Cancer Res. 61:8375-80]. Low amplification (20 cycles) was used to prevent overamplification of unrestricted repetitive sequences in the ligated DNA and yet yield sufficient PCR products for single or low-copy number CGI loci. Cy5-labeled amplicons, representing pools of methylated DNA fragments derived from tumors and normal controls, were co-hybridized with a Cy3-labeled common reference amplicon to the CGI microarrays. In analogy to gene expression profiling, we chose a common reference design to allow comparison of methylation across all tumor and normal samples. The common reference consisted of a pool of six CRC cell lines (SW48, RKO, SW480, Caco2, SW837, LS411). Detection was done on a G2565BA scanner (Agilent Technologies), and image analysis using GenePix6.0 (Molecular Devices).

### Analysis of CGI Microarrays

Preprocessing and statistical analysis was performed in Rosetta Resolver (Rosetta Biosoftware) using custom R plug-ins for error-weighted ANOVA with multiple testing correction [Hochberg and Benjamini (1990), Stat. Med. 9:811-8].

### Bisulphite Validation Methods

Patient DNA samples (500 ng) were converted using the EZ DNA methylation Gold bisulfite kit (Zymo Research). Primers for bisulfite sequence analysis (BSA) of the PTPRG intron 1 locus were designed using MethPrimer [Li and Dahiya (2002), Bioinformatics 18:1427-31]. Left primer 5′-GATTTAAAATGGGATTGTTAGGGAAT-3′ (SEQ ID NO:9) and right primer 5′-CTTACTCCAAAACCTACTACTCCCTCT-3′ (SEQ ID NO:10). For clonal BSA, up to ten colonies of cloned bisulfite PCR product were sequenced for both normal and tumor. The resulting PCR product (224 bp) was sequenced using the right primer. The same specific primer sequences were extended with T7 RNA polymerase promoter sequences for base-specific cleavage and mass spectrometry analysis [Ehrich et al. (2005), Proc. Natl. Acad. Sci. U.S.A. 102:15785-15790].

### Results

### CpG Island Methylation Profiling of Colorectal Tumors

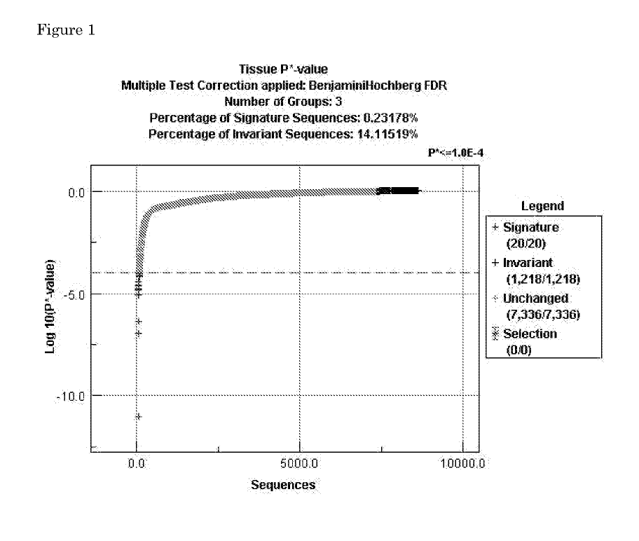

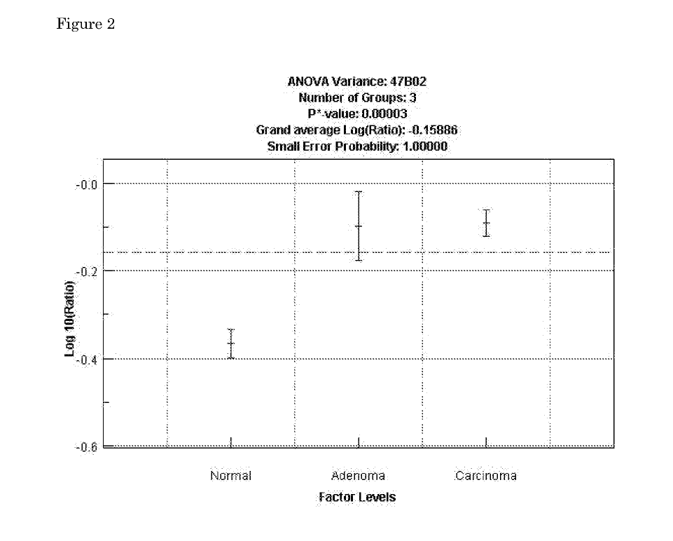

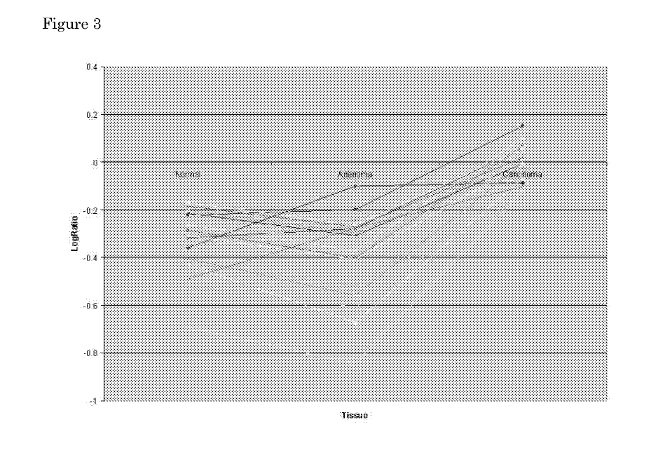

CpG island microarray profiling was used for the high-throughput analysis of methylation status of 8.6 K CpG islands in 17 right-sided carcinomatous, two adenomatous and five corresponding normal colonic epithelium samples. The microarray data were tested for differential methylation between tumors (including both carcinomas and adenomas) and normal samples using error-weighted analysis of variance. We identified 22 loci with a false discovery rate (FDR)<1%. In addition, an ANOVA for the three histology groups was performed, although the adenoma group contained only two samples. In this analysis, we identified 20 loci with a very stringent FDR<0.01% (FIG. 1). The most significant CpG island clone in both analyses was 47B02, which mapped to the first intron of the PTPRG gene. The log ratios of tumor and adenoma samples compared to the CRC cell line reference panel are close to 0, indicating comparable methylation levels in the primary tumors and the CRC cell lines. The normal samples showed negative log ratios, indicating that the intron 1 PTPRG CGI was hypermethylated in both carcinomas and the adenomas compared to normal colon (FIG. 2). This finding indicates that the methylation of the PTPRG locus could be an early event in tumorigenesis. The remaining selected loci were hypermethylated in a high percentage of carcinomas, but not in the tested adenomas and normal colon samples (FIG. 3).

### Example 2

### Validation and Extension of Array Data Using Bisulphite Sequence Analysis

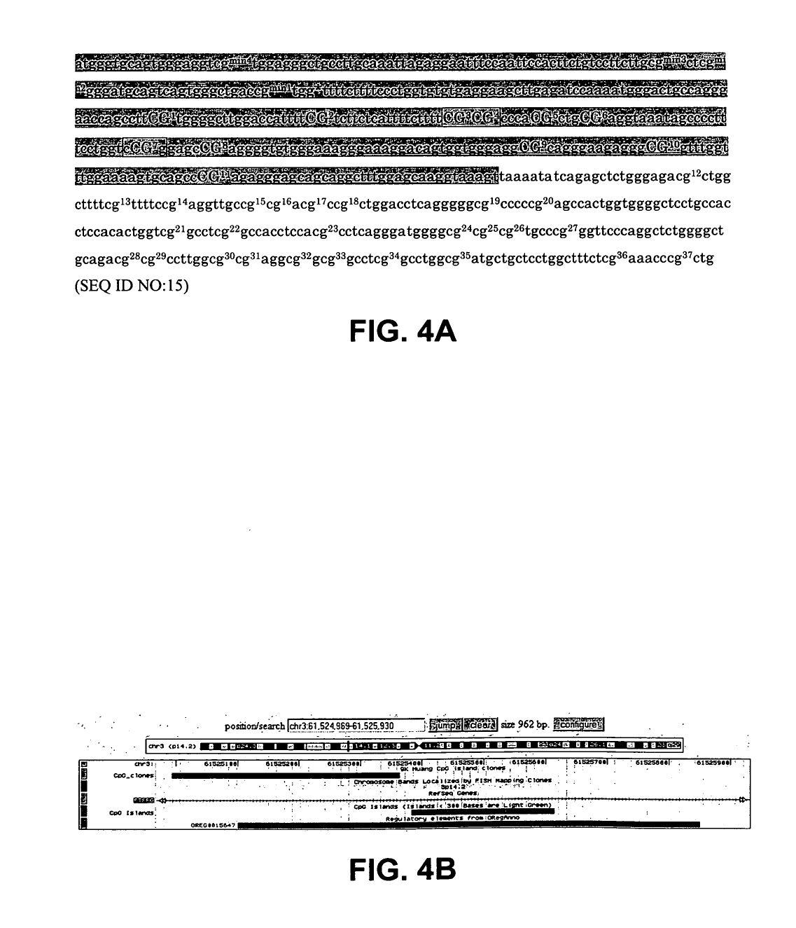

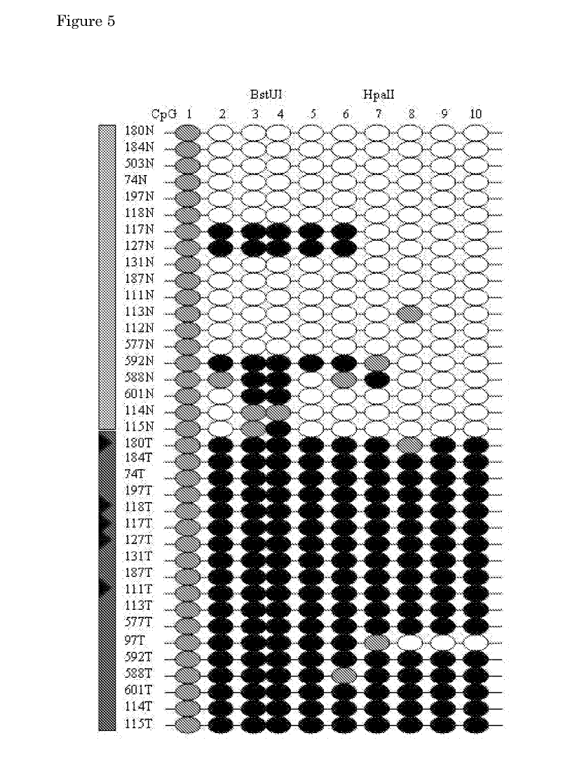

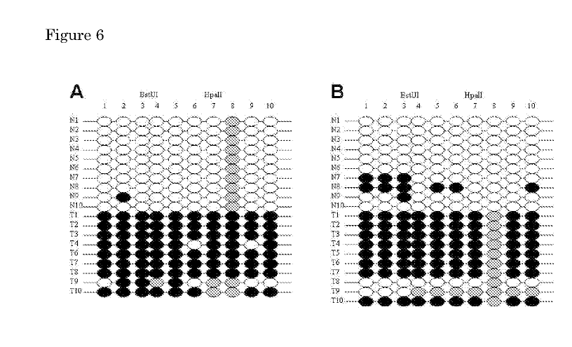

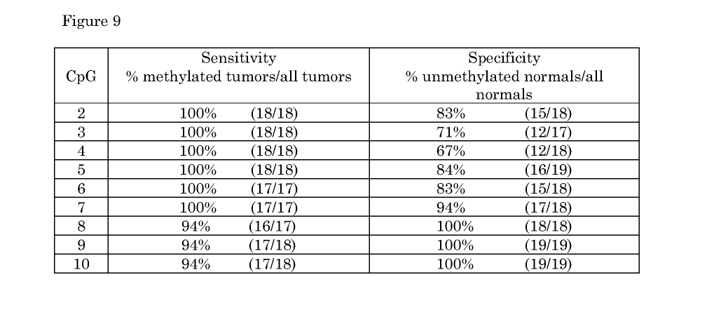

The PTPRG intron 1 methylation status was validated using direct bisulphite sequence analysis (BSA). Part of the 47B02 clone sequence was used to design BSA primers (FIG. 4). Additional right-sided adenoma (two) and carcinoma (seven) samples were included as well as two left-sided tumors (one adenoma, one carcinoma). For all but one tumor, the methylation status of paired normal samples was determined (FIG. 5). The selected amplified region contains two methylation-sensitive restriction sites that were used in the amplicon generation for the differential methylation hybridization. BSA gives a resolution of single CpGs; CpGs 2 through 10 could be evaluated using this method. All of the 18 tumor samples showed methylation of the region, while one carcinoma showed partial methylation. In contrast, the normal samples were mostly unmethylated, with six samples showing partial methylation of one to five CpGs. CpGs 7-10 showed the best distinction between tumor and normal in this set of samples. The tumor-specific methylation frequency of this locus is very high; based on the current data set we could estimate a sensitivity of 94-100% to detect methylation in adenoma/carcinoma tissue and a specificity of 94-100% for CpGs 7-10 (see FIG. 9). Therefore, the methylation microarray results were confirmed and extended to additional proximal and distal adenomas and carcinomas.

To study the methylation of the PTPRG intron 1 locus at the single chromosome level, clonal bisulphite analysis was performed on four pairs of tumor-normal samples. Clonal BSA confirmed the direct BSA results, and showed that in the partially methylated normal samples, at most three out of ten alleles were methylated for several CpGs (FIG. 6). Since the percentage of tumor cells varied between 30% (T74) and 80% in the tumor biopsies, as expected, we find some alleles unmethylated in the sequenced tumor clones.

### Example 3

### Quantitative Assessment of PTPRG Intron 1 Methylation

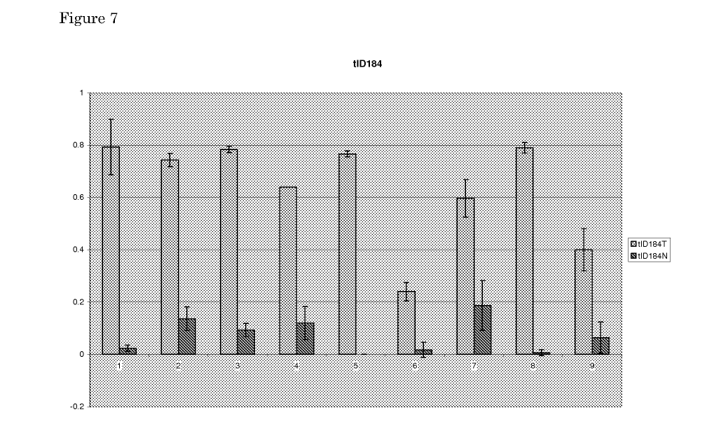

To further confirm our findings, we used a quantitative high-throughput analysis of PTPRG intron 1 methylation patterns by base-specific cleavage and mass spectrometry [Ehrich et al. (2005), Proc. Natl. Acad. Sci. U.S.A. 102:15785-15790] on nine previously analyzed tumor-normal pairs. A representative example result for tID184 illustrates tumor-specific methylation of all fragments, covering 11 CpG dinucleotides (FIG. 7). Fragment 6 gave overall low quality results because of its small size. The other eight tumor-normal pairs also confirmed tumor-specific hypermethylation of all the fragments present in the amplified PTPRG locus. A percentage between 35-80% methylation of tumor fragments was found, which correlated well with the estimated percentage of tumor cells in the analyzed tissue.

Therefore, we identified and confirmed tumor-specific methylation, most specifically for four CpGs in the 47B02 clone locus in the first intron of PTPRG, in 94% of tested carcinomas and adenomas from different locations in the colon and rectum. Initial analysis of expression of the main isoform of PTPRG mRNA by quantitative RT-PCR did not show a consistent effect, however a small reduction in expression associated with methylation of the intron 1 locus as well as decreased expression of alternative transcripts cannot be ruled out.

### Example 4

### Methods

### MS-MLPA Probe Design and Assay

Custom MS-MLPA probes for the PTPRG locus were designed in primer3 [Rozen and Skaletsky (2000), Methods Mol. Biol. 132:365-386] and included CpG 9 and 10 (as numbered in FIG. 4A) in the sequence investigated by BSA. Probes used are: PTPRG L: 5′-GAAAGGACAGTGGTGGGAGGC-3′ (SEQ ID NO:11) (Tm 63.9° C.) and PTPRG_R: 5′-GCAGGGAAGAGGGCGGTT-3′ (SEQ ID NO:12) (Tm 63.36° C.), genomic region Chr3: 61525269-61525308 (UCSC assembly: March 2006).

As a control, we have used a BRCA2 probeset from the MRC-Holland SALSA MS-MLPA KIT ME001B Tumor suppressor-1 kit: BRCA2_L: 5′-GGCCATGGAATCTGCTGAACAAAA-3′ (SEQ ID NO:13) and BRCA2_R: 5′-GGAACAAGGTTTATCAAGGGATGTCACAACCGTGTGGAAGTTGCG-3′ (SEQ ID NO:14); genomic region Chr13: 31851549-31851617 (UCSC assembly: March 2006). Fragment analysis was performed on an ABI 3130 (Applied Biosystems, Foster City, US). MLPA reagents were obtained from MRC-Holland, Amsterdam, The Netherlands (EK1 kit; www.mlpa.com). Approximately 50 ng of genomic DNA in 5 μl of water was used as input. The MS-MPLA was performed as described by Nygren et al [Nygren et al. (2005), Nucleic Acids Res. 33:e128]. Negative (human semen DNA) and 100% methylated DNA controls (CpGenome Universal methylated DNA, Chemicon (Millipore), Billerica, Mass., USA), were included every run to asses HhaI cleavage.

### MS-MLPA Analysis

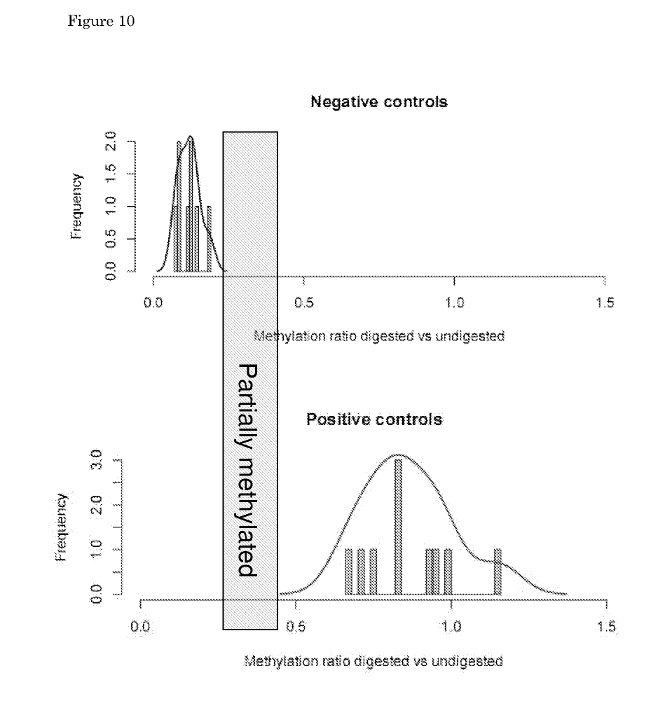

Fragment run analysis was performed in Genemapper (Applied Biosystems, Foster City, US). Peak heights were exported and processed in Excel. PTPRG peak heights were normalized by division with the BRCA2 peak heights of the same run. Subsequently, the ratio of the HhaI-digested reaction was divided by the ratio of the undigested reaction providing one ratio per sample. Ten individual measurements of the unmethylated DNA and fully methylated control DNAs provided a ratio distribution for each. Tested samples falling within three standard deviations of the mean of the unmethylated and methylated reference samples were typed accordingly. Samples containing ratios between these standard deviation boundaries were typed partially methylated (FIG. 10).

### Results

### MS-MLPA Validation of PTPRG Intron 1 Methylation

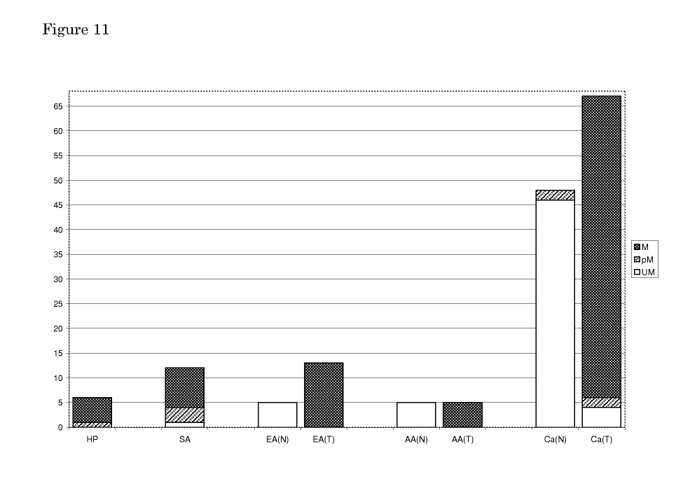

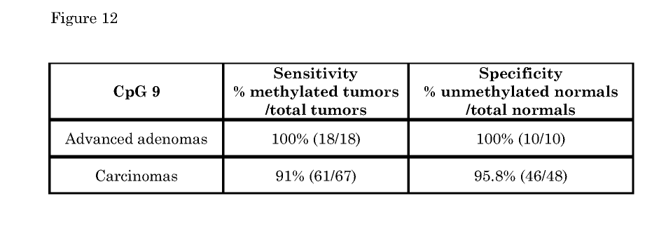

We developed a methylation-specific multiplex ligation-dependent probe amplification (MS-MLPA) assay to test methylation of specific CpGs in the 3′ region of the BSA-validated region in a consecutive CRC validation series (FIG. 11). The MS-MLPA assay is robust and can be performed on DNA derived from formalin-fixed paraffin-embedded tissues. Of the 67 tested carcinoma samples, 91% showed (partial) methylation of the targeted CpG dinucleotides, while 95.8% of the corresponding normal mucosa samples was unmethylated (n=48, FIG. 12). Even higher levels of sensitivity and specificity were obtained for advanced adenomas (FIG. 12). A few lesions preceding the adenoma/carcinoma stages were tested for which no corresponding normal tissue was available. Sensitivities of 83.3% for hyperplastic polyps (n=6) and 66.7% for serrated adenomas (n=12) were obtained (FIG. 11). PTPRG intron 1 CpG9 was also methylated in sporadic mismatch repair-deficient colon tumors due to MLH1 promoter methylation and, surprisingly, in all 11 colon cancers tested from patients suffering from the Lynch syndrome with germline mutations in one of the MMR genes.

Genomic Locus Overlaps with Regulatory Feature

Overlapping with the 47B02 sequence, a regulatory feature has been annotated in the human genome (OREG0015647, chr3: 61,525,101-61,525851 of UCSC March 2006 assembly) based on chromatin immunoprecipitation-on-chip studies for binding sites of the vertebrate insulator protein CTCF [Kim et al. (2007), Cell 128:1231-45]. Most of the experimentally discovered CTCF binding sites in this study are located far from the transcriptional start sites, with their distribution strongly correlated with genes. Also, CTCF binding sites are largely invariant across different cell types [Kim et al. (2007), Cell 128:1231-45].

Recent studies have identified CTCF to be the vertebrate insulator protein [Bell et al. (1999), Cell 98:387-396]. Insulator elements affect gene expression by preventing the spread of heterochromatin and restricting transcriptional enhancers from activation of unrelated promoters. So far, CTCF remains as the only major protein implicated in establishment of insulators in vertebrates [Felsenfeld et al. (2004), Cold Spring Harb., Symp. Quant. Biol. 69:245-250], including those involved in regulation of gene imprinting and monoallelic gene expression [Fedoriw et al. (2004), Science 303:238-240.; Ling et al. (2006), Science 312:269-272], as well as in X chromosome inactivation and in the escape from X-linked inactivation [Filippova et al. (2005), Dev. Cell. 8:31-42; Lee (2003), Curr. Biol. 13:R242-R254]. The mechanism of insulator function remains unclear. One model proposes that insulators, by formation of special chromatin structures, compete for enhancer-bound activators, preventing the activation of downstream promoters [Bulger and Groudine (1999), Genes Dev. 13:2465-2477]. Alternatively, insulators may facilitate the formation of loops, for example, via attachment of chromosomal regions to the nuclear membrane [Yusufzai et al. (2004), Mol. Cell. 13:291-298], keeping the intermediate regions exposed for only local interactions between enhancers and promoters. Consistent with this model, it was recently shown that CTCF could mediate long-range chromosomal interactions in mammalian cells, providing a possible mechanism by which insulators establish regulatory domains [Kurukuti et al. (2006), Proc. Natl. Acad. Sci. U.S.A. 103:10684-10689; Ling et al. (2006), Science 312:269-272; Yusufzai et al. (2004), Mol. Cell. 13:291-298]. Methylation of several CTCF binding sites was shown to abolish CTCF binding [Bell et al. (2000), Nature 405:482-485; Filippova et al. (2005), Dev. Cell. 8:31-42; Hark et al. (2000), Nature 405:486-489; Kanduri et al. (2000), Curr. Biol. 10:853-856; Mukhopadhyay et al. (2004), Genome Res. 14:1594-1602].

We performed chromatin immunoprecipitation with an antibody against human CTCF on normal human fibroblast chromatin. In the bound fraction, we amplified the MS-MLPA product MLPA2 (double underline in FIG. 4A). The fragment was not amplified after HhaI digestion, indicating that CpG9 is unmethylated in normal fibroblasts. Therefore, in the overlapping region between clone 47B02 and OREG0015647, we experimentally confirmed binding of CTCF protein to a 39 bp region covered by the MLPA2 primers in fibroblasts. These 39 bp lie within the most tumor-specific methylated region.

1.-24. (canceled)

25. A method for determining whether a subject is suffering from a tumor in a tissue, the method comprising:
determining, from a sample comprising nucleic acid from the tissue, the methylation of a CpG in the genomic region of the receptor protein-tyrosine phosphatase gamma gene and
determining from the methylation thus determined whether the subject is suffering from a tumor in the tissue.

26. The method according to claim 25, wherein one end of the genomic region is at one (1) mega base pairs upstream from the transcription initiation site at chr3:61,522,283 (March 2006 human reference sequence (NCBI Build 36.1)) and a second end of the genomic region is at one (1) mega base pairs downstream from the polyadenylation site at chr3:62,255,613 of the gene.

27. The method according to claim 25, wherein the CpG is present in the receptor protein-tyrosine phosphatase gamma gene's first intron.

28. The method according to claim 25, wherein the CpG comprises at least one of CpG indicated in FIG. 4.

29. The method according to claim 28, wherein the CpG is selected from the group consisting of CpG1, CpG2, CpG3, CpG4, CpG5, CpG6, CpG7, CpG8, CpG9, CpG 10, and CpG11 indicated in FIG. 4.

30. A kit for determining whether a subject suffers from a tumor, the kit comprising:
means for determining the methylation of a CpG present in a genomic region of the subject's receptor protein-tyrosine phosphatase gamma gene, and including a region up to one (1) mega base pairs upstream from the transcription initiation site thereof and one (1) mega base pairs downstream from the polyadenylation site of the receptor protein-tyrosine phosphatase gamma gene, in a sample comprising nucleic acid from the person, and
a container for the sample.

31. The kit of claim 30, further comprising:
means for amplifying a qCpG that is present in the receptor protein-tyrosine phosphatase gamma gene's first intron.

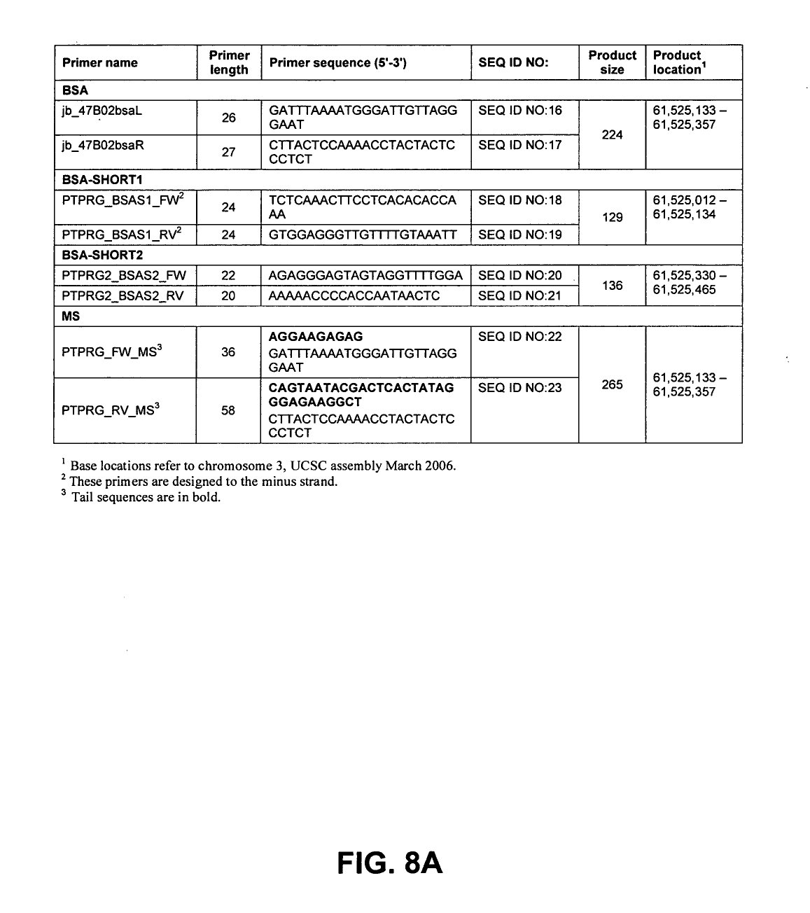

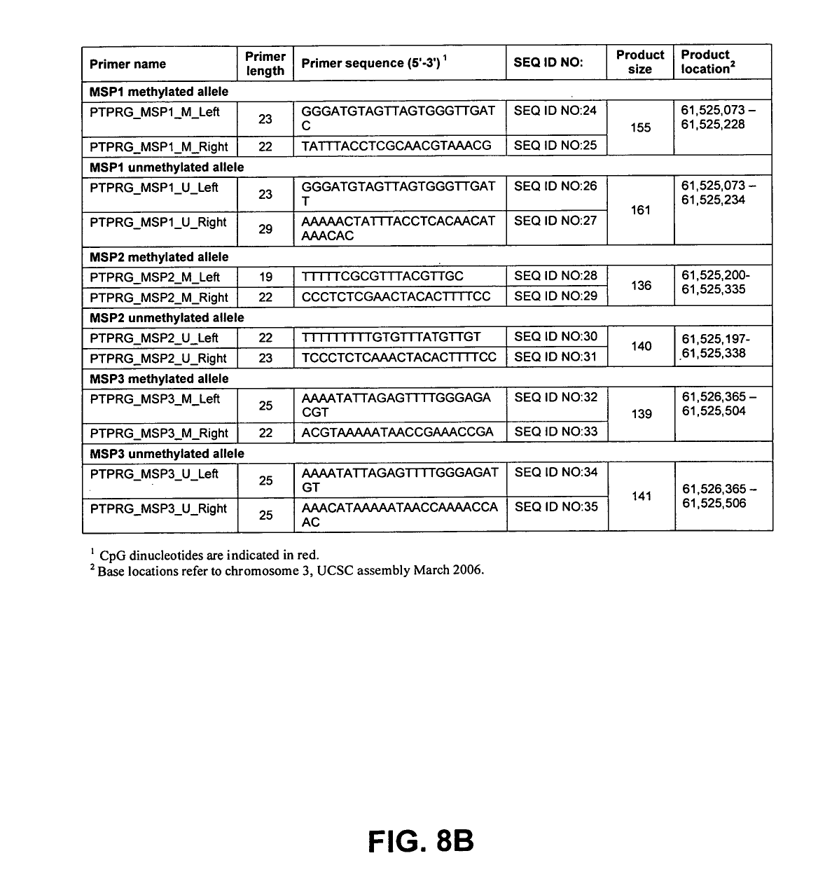

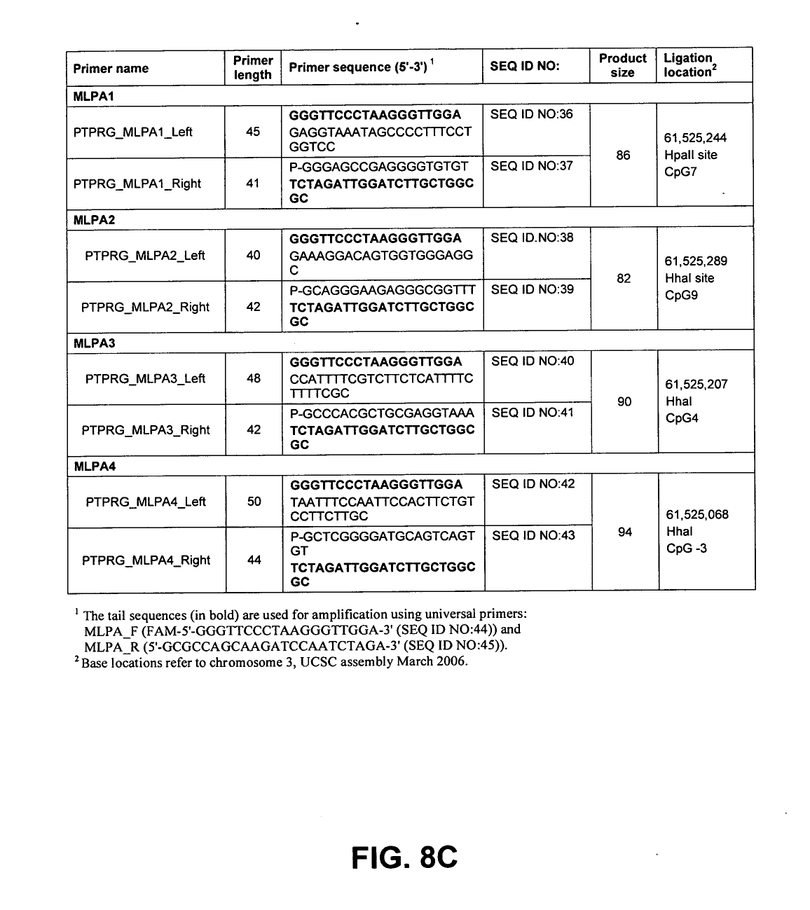

32. The kit of claim 30, comprising a set of primers as indicated in FIG. 8.

33. A method for determining whether a subject is suffering from a tumor in a tissue, the method comprising:
determining, from a sample containing nucleic acid from the tissue, an mRNA expression level of the receptor protein-tyrosine phosphatase gamma gene, and
determining from the mRNA expression level thus determined whether the subject is suffering from a tumor in the tissue.

34. The method according to claim 33, further comprising:
comparing the determined expression level of the receptor protein-tyrosine phosphatase gamma gene to the expression level of the gene in a reference sample.

35. A method for determining whether a subject is suffering from a tumor in a tissue, the method comprising:
determining, from a sample containing protein from the tissue, a protein expression level of the receptor protein-tyrosine phosphatase gamma gene product, and
determining from the expression level thus determined whether the subject is suffering from a tumor in the tissue.

36. The method according to claim 35, further comprising:
comparing the determined expression level of the receptor protein-tyrosine phosphatase gamma gene to the expression level of the gene in a reference sample.

37. A method for determining whether a subject is suffering from a tumor in a tissue, the method comprising:
determining, in a sample comprising nucleic acid of the genomic region of the receptor protein-tyrosine phosphatase gamma gene from the tissue, whether the CCCTC-binding factor (zinc finger protein) can bind to nucleic acid of the genomic region of the receptor protein-tyrosine phosphatase gamma gene.

38. A method for determining whether a subject is suffering from a tumor in a tissue, the method comprising:
determining, in a sample comprising nucleic acid of the first intron of the receptor protein-tyrosine phosphatase gamma gene from the tissue, whether the CCCTC-binding factor (zinc finger protein) can bind to nucleic acid of the first intron.

39. A method for determining whether a sample of a tissue of a subject comprises tumor cells, the method comprising:
determining whether CTCF protein can bind to nucleic acid of the first intron of the receptor protein-tyrosine phosphatase gamma gene from the tissue.
